(19) United States
(12) Patent Application Publication
(10) Pub. No.: US 2026/0263805 A1
(43) Pub. Date: Sep. 10, 2026

(54) Systems and Methods For Affecting Dysfunction With Stimulation

(71) Applicant: Cochlear Limited, New South Waies (AU)

(72) Inventors: Remo Albert Gerardus Joseph Arts, Valkenswaards (NL); Kelly Assouly, Schaerbeek (BE)

(73) Assignee: Cochlear Limited, Macquarie University, NSW (AU)

(21) Appl. No.: 19/166,836

(22) PCT Filed: Mar. 26, 2024
(86) PCT No.: PCT/IB2024/052916

§ 371 Date: Sep. 18, 2025

Related U.S. Application Data
(60) Provisional application No. 63/457,248, filed on Apr. 5, 2023.

(51) Int. Cl. A61N 1/36

(52) U.S. Cl. CPC A61N 1/361; A61N 1/36038

(57) ABSTRACT

A system includes an electrical stimulator configured to provide at least one asymmetric multiphasic stimulation to a recipient for affecting tinnitus in the recipient. A method includes generating asymmetric multiphasic stimulation. The method can also include providing the asymmetric multiphasic stimulation to an ear of a recipient to affect tinnitus in the recipient.

### CROSS REFERENCE TO RELATED APPLICATION

This patent application claims priority to U.S. provisional patent application 63/457,248, filed Apr. 5, 2023, which is incorporated by reference herein in its entirety.

### TECHNICAL FIELD

The present disclosure relates to systems and methods for affecting dysfunction with stimulation.

### BACKGROUND OF THE INVENTION

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY OF THE INVENTION

According to a first embodiment disclosed herein, a system includes a stimulator configured to provide at least one asymmetric multiphasic stimulation to a recipient for affecting tinnitus in the recipient.

According to a second embodiment disclosed herein, a method includes generating asymmetric multiphasic stimulation; and providing the asymmetric multiphasic stimulation to an ear of a recipient to affect tinnitus in the recipient.

According to a third embodiment disclosed herein, a non-transitory computer readable storage medium includes computer readable instructions stored thereon for causing a computing system to: provide cathodic stimulus comprising first non-symmetric pulses to a recipient; provide anodic stimulus comprising second non-symmetric pulses to the recipient; and compare an effect of the cathodic stimulus with an effect of the anodic stimulus on the recipient.

According to a fourth embodiment disclosed herein, a system includes an electrical stimulator; a stimulation controller configured to cause the electrical stimulator to deliver an electrical non-symmetric multiphasic stimulus to a recipient; and a measurement sensor configured to measure a potential evoked in the recipient in response to the electrical non-symmetric multiphasic stimulus.

### BRIEF DESCRIPTION OF DRAWINGS

FIG. 1A depicts a schematic diagram of an exemplary cochlear implant that can be configured to implement aspects of the techniques presented herein, according to some exemplary embodiments.

FIG. 1B is a block diagram of the cochlear implant system of FIG. 1A, according to an embodiment.

FIGS. 2A-2H are diagrams that illustrate examples of 8 types of electrical stimulation that can be provided to a recipient to achieve various diagnostic and therapeutic effects, as disclosed in further detail herein.

FIG. 3 is a diagram that depicts an example of a system for evaluating and treating dysfunction in a recipient, according to an embodiment.

FIG. 4 depicts a flow chart that illustrates examples of operations that can be performed to test, diagnose, and provide treatment to a recipient using asymmetric multiphasic stimulation.

FIG. 5 depicts a graphical diagram that shows a process for providing multiphasic stimulation to a recipient for assessing and treating a dysfunction in the recipient.

FIG. 6 depicts a flow chart that illustrates other examples of operations that can be performed to test, diagnose, and treat a recipient for a dysfunction using asymmetric multiphasic stimulation.

FIG. 7 illustrates an example of a computing system within which one or more of the disclosed embodiments can be implemented.

### DETAILED DESCRIPTION

Merely for ease of description, the techniques presented herein are primarily described herein with reference to an illustrative medical device, namely a cochlear implant. However, it is to be appreciated that the techniques presented herein may also be used with a variety of other devices that provide a wide range of benefits to recipients, patients, or other users of the devices. As examples, the techniques presented herein can be used in or with consumer electronics, Internet-of-Things (IoT) devices, wireless devices, audio equipment, sound processing devices, computing systems (e.g., servers in data centers), networking devices, and various types of software systems, such as databases, machine learning and artificial intelligence systems, etc. As other examples, the techniques presented herein may be used in or with medical devices such as cochlear implants and other hearing prostheses, including acoustic hearing aids, bone conduction devices, middle ear auditory prostheses, direct acoustic stimulators, other electrically stimulating auditory prostheses (e.g., auditory brain stimulators), etc. The techniques presented herein may also be used in or with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc. In further embodiments, the techniques presented herein may be used in or with air purifiers or air sensors (e.g., automatically adjust depending on environment), hospital beds, identification (ID) badges/bands, or other hospital equipment or instruments.

The teachings detailed herein can be implemented in or with sensory prostheses, such as hearing implants specifically, and neural stimulation devices in general. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. Moreover, with respect to any teachings herein, such corresponds to a disclosure of utilizing those teachings with a cochlear implant, a bone conduction device (active and passive transcutaneous bone conduction devices, and percutaneous bone conduction devices) and a middle ear implant, providing that the art enables such, unless otherwise noted. To be clear, any teaching herein with respect to a specific sensory prosthesis corresponds to a disclosure of utilizing those teachings in/with any of the aforementioned hearing prostheses, and visa-versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other devices or prostheses noted herein, whether a species of a hearing prosthesis, or a species of a sensory prosthesis, such as a retinal prosthesis. In this regard, any disclosure herein with respect to evoking a hearing percept corresponds to a disclosure of evoking other types of neural percepts in other embodiments, such as a visual/sight percept, a tactile percept, a smell precept or a taste percept, unless otherwise indicated and/or unless the art does not enable such. Any disclosure herein of a device, system and/or method that is used to or results in ultimate stimulation of the auditory nerve corresponds to a disclosure of an analogous stimulation of the optic nerve utilizing analogous components, methods, and/or systems.

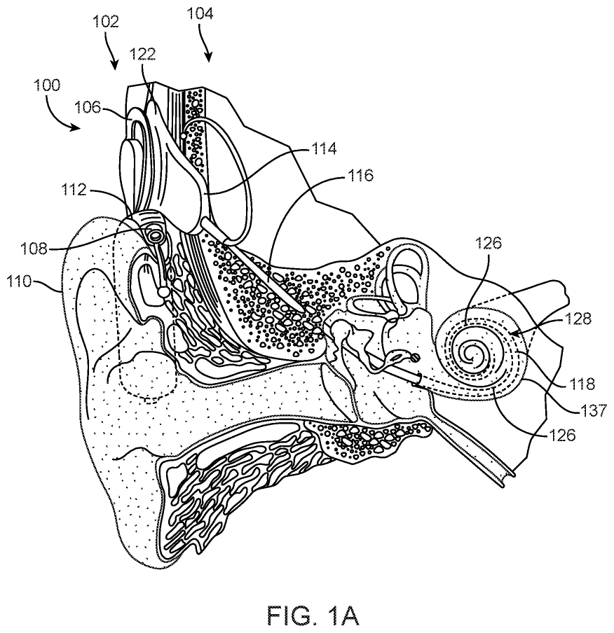

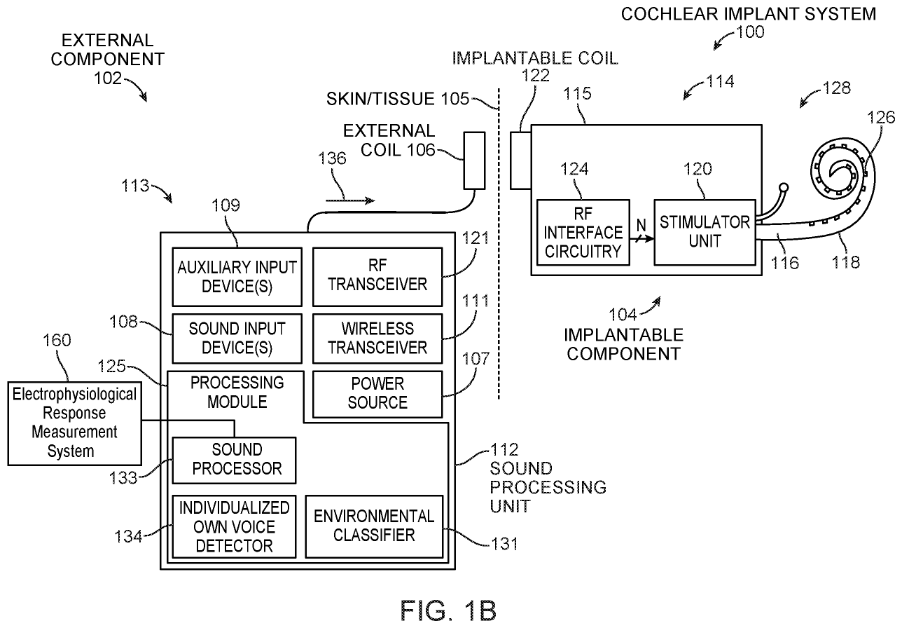

FIG. 1A is a schematic diagram of an exemplary cochlear implant system 100 configured to implement aspects of the techniques presented herein. FIG. 1B is a block diagram of the cochlear implant system 100 of FIG. 1A. For ease of illustration, FIGS. 1A and 1B are described together herein. The cochlear implant system 100 includes an external component 102 and an internal/implantable component 104. The external component 102 is directly or indirectly attached to the body of the recipient and typically comprises an external coil 106 and, generally, a magnet (not shown in FIGS. 1A-1B) fixed relative to the external coil 106. The external component 102 also comprises one or more input elements/devices 113 (shown in FIG. 1B) for receiving input signals at a sound processing unit 112. In this example, the one or more input devices 113 include sound input devices 108 (e.g., microphones positioned by auricle 110 of the recipient, telecoils, etc.) configured to capture/receive input signals, one or more auxiliary input devices 109 (e.g., audio ports, such as a Direct Audio Input (DAI), data ports, such as a Universal Serial Bus (USB) port, cable port, etc.), and a wireless transmitter/receiver (transceiver) 111, each located in, on, or near the sound processing unit 112.

The sound processing unit 112 also includes, for example, at least one power source 107, a radio-frequency (RF) transceiver 121, and a processing module 125. The processing module 125 includes a number of elements, including an environmental classifier 131, a sound processor 133, and an individualized own voice detector 134. Each of the environmental classifier 131, the sound processor 133, and the individualized own voice detector 134 can be formed by one or more processors (e.g., one or more Digital Signal Processors (DSPs), one or more processing cores, etc.), firmware, software, etc. arranged to perform operations described herein. That is, the environmental classifier 131, the sound processor 133, and the individualized own voice detector 134 can each be implemented as firmware elements, partially or fully implemented with digital logic gates in one or more application-specific integrated circuits (ASICs), partially or fully in software, etc.

In the examples of FIGS. 1A and 1B, the sound processing unit 112 is a behind-the-ear (BTE) sound processing unit configured to be attached to, and worn adjacent to, the recipient's ear. However, it is to be appreciated that sound processing unit 112 can have other arrangements, such as an off the ear (OTE) processing unit (e.g., a component having a generally cylindrical shape and that is configured to be magnetically coupled to the recipient's head), etc., a mini or micro-BTE unit, an in-the-canal unit that is configured to be located in the recipient's ear canal, a body-worn sound processing unit, etc.

In the exemplary embodiment of FIGS. 1A and 1B, the implantable component 104 includes an implant body (main module) 114, a lead region 116, and an intra-cochlear stimulating assembly 118, all configured to be implanted under the skin/tissue (tissue) 105 of the recipient. The implant body 114 generally includes a hermetically-sealed housing 115 in which RF interface circuitry 124 and a stimulator unit 120 are disposed. The implant body 114 also includes an internal/implantable coil 122 that is generally external to the housing 115, but that is connected to the RF interface circuitry 124 via a hermetic feedthrough (not shown in FIG. 1B).

Stimulating assembly 118 is configured to be at least partially implanted in the recipient's cochlea 137. Stimulating assembly 118 includes a plurality of longitudinally spaced intra-cochlear electrical stimulating contacts (e.g., electrodes) 126 that collectively form a contact or electrode array 128 for delivery of electrical stimulation (current) to the recipient's cochlea. Stimulating assembly 118 extends through an opening in the recipient's cochlea (e.g., cochleostomy, the round window, etc.) and has a proximal end connected to stimulator unit 120 via lead region 116 and a hermetic feedthrough (not shown in FIG. 1B). Lead region 116 includes a plurality of conductors (wires) that electrically couple the stimulating contacts 126 to the stimulator unit 120.

As noted, the cochlear implant system 100 includes the external coil 106 and the implantable coil 122. The coils 106 and 122 are typically wire antenna coils each comprised of multiple turns of electrically insulated single-strand or multi-strand wire. Generally, a magnet is fixed in position relative to each of the external coil 106 and the implantable coil 122. In some embodiments, the external component 102 and/or the implantable component 104 can include magnet assemblies that each have more than one magnetic component. The magnets fixed relative to the external coil 106 and the implantable coil 122 facilitate the operational alignment of the external coil with the implantable coil. This operational alignment of the coils 106 and 122 enables the external component 102 to transmit data, as well as possibly power, to the implantable component 104 via a closely-coupled wireless link formed between the external coil 106 and the implantable coil 122. In certain examples, the closely-coupled wireless link is a radio frequency (RF) link. However, various other types of energy transfer, such as infrared (IR), electromagnetic, capacitive and inductive transfer, can be used to transfer the power and/or data from an external component to an implantable component and, as such, FIG. 1B illustrates only one exemplary arrangement.

As noted above, sound processing unit 112 includes the processing module 125. The processing module 125 is configured to convert input audio signals into stimulation control signals 136 for use in stimulating a first ear of a recipient (i.e., the processing module 125 is configured to perform sound processing on input audio signals received at the sound processing unit 112). Stated differently, the sound processor 133 (e.g., one or more processing elements implementing firmware, software, etc.) is configured to convert the captured input audio signals into stimulation control signals 136 that represent electrical stimulation for delivery to the recipient. The input audio signals that are processed and converted into stimulation control signals 136 can be audio signals received via the sound input devices 108, signals received via the auxiliary input devices 109, and/or signals received via the wireless transceiver 111.

In the embodiment of FIG. 1B, the stimulation control signals 136 are provided to the RF transceiver 121, which transcutaneously transfers the stimulation control signals 136 (e.g., in an encoded manner) to the implantable component 104 via external coil 106 and implantable coil 122. The stimulation control signals 136 are received at the RF interface circuitry 124 via implantable coil 122 and provided to the stimulator unit 120 (e.g., as an N number of signals). The stimulator unit 120 is configured to utilize the stimulation control signals 136 to generate electrical stimulation signals (e.g., current signals) for delivery to the recipient's cochlea via one or more stimulating contacts 126 (e.g., electrode) in array 128. In this way, cochlear implant system 100 electrically stimulates the recipient's auditory nerve cells, bypassing absent or defective hair cells that normally transduce acoustic vibrations into neural activity, in a manner that causes the recipient to perceive one or more components of the input audio signals.

FIG. 1B also illustrates an electrophysiological response measurement system 160 that is communicably coupled to the sound processor 133 via a connection (e.g., a cable). The electrophysiological response measurement system 160 is, in some embodiments, a processor-based system such as a personal computer, server, workstation or the like, having one or more processors that execute software programs to perform various techniques disclosed herein. For example, system 160 can generate a signal that is used by the cochlear implant system 100 as a stimulus to stimulate the auditory nerve of the recipient via one or more stimulating contacts 126, receive a measurement of neural activity in response to the stimulus from the cochlear implant system 100, and evaluate the measurement of the neural activity, as disclosed in further detail herein.

Tinnitus is a kind of auditory dysfunction that includes experiencing the perception of sound in the absence of an external stimuli. For example, tinnitus may be experienced as a “ringing” in the ears. Tinnitus is a common artefact of hearing loss, but tinnitus may also be a symptom of other underlying conditions, such as ear injuries, circulatory system disorders, etc. Tinnitus can, for example, be caused by abnormal hair cells in the cochlea of an ear generating erroneous signals. Tinnitus is an auditory phantom perception, which may be perceived as having various characteristics (e.g., pure tone; narrow band noise; polyphonic), and is experienced either unilaterally, bilaterally, or in the head. In some cases, the perception of tinnitus is intermittent or variable in magnitude.

Although tinnitus effects can range from mild to severe, almost one-quarter of those individuals with tinnitus describe their tinnitus as disabling or nearly disabling. In some individuals, tinnitus can be disabling and incapacitating, deteriorating quality of life, including sleep quality. Hearing loss is a common condition associated with tinnitus.

Masking has been used to treat tinnitus, with either acoustic or electrical stimulation. Masking can comprise adding an audible or inaudible masking stimulus (e.g., signals) corresponding to sound (e.g., white noise; music; patterned sound; low-level sound; sound tailored based on characteristics of the recipient's tinnitus) intended to mask or cover up a phantom sound (e.g., ringing; hissing) caused by tinnitus. The added sound level can be close to, softer than, or louder than the perceived loudness of the phantom sound. While the tinnitus can be partially or fully masked by the added audible or inaudible sound such that the recipient's perception of the phantom sound is reduced, masking does not reduce or eliminate the tinnitus itself. In addition, some individuals may find the fitting procedure for a tinnitus device to be uncomfortable, particularly if prolonged conscious attention to one's tinnitus is required as the device is being adjusted.

Electrical stimulation of the auditory nerve can improve hearing perception, such as speech perception and sound localization. Electrical stimulation of the auditory nerve may also provide tinnitus relief. However, the effects of electrical stimulation of the auditory nerve varies across recipients. Direct current stimulation can damage the tissue of a recipient, while charge-balanced electrical pulses typically do not cause tissue damage in recipients. Therefore, biphasic electrical pulses that are charge-balanced are typically used in cochlear implant systems to generate the perception of sound in a recipient.

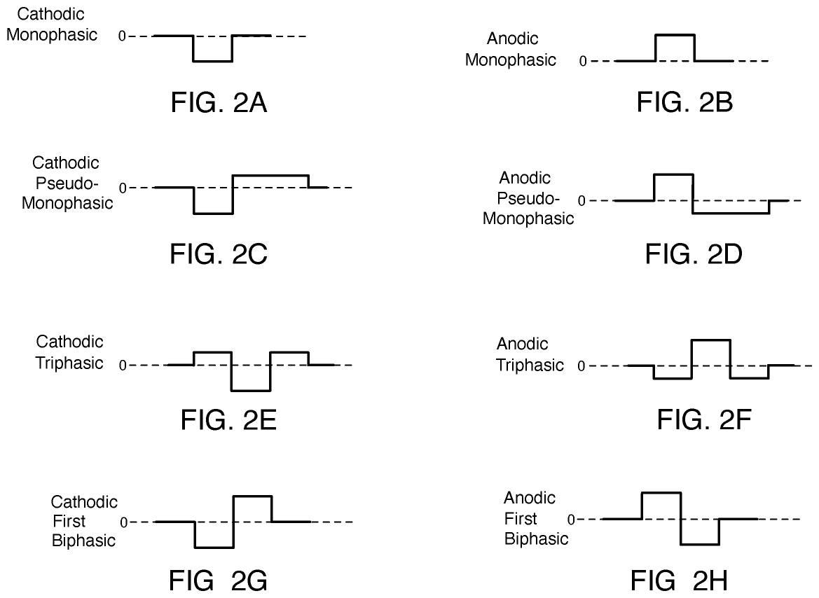

FIGS. 2A-2H are diagrams that illustrate examples of 8 types of electrical stimulation that can be provided to a recipient to achieve various diagnostic and therapeutic effects, as disclosed in further detail herein. The 8 types of electrical stimulation are shown in FIGS. 2A-2H as examples that are provided for illustrative and comparison purposes and are not intended to be limiting. The 8 types of electrical stimulation shown in FIGS. 2A-2H include cathodic and anodic pulses of electrical current. Cathodic pulses are shown as negative pulses (below zero) in FIGS. 2A-2H, and anodic pulses are shown as positive pulses (above zero) in FIGS. 2A-2H. The cathodic and anodic pulses are shown as ideal rectangular waveforms in FIGS. 2A-2H as examples. However, it should be understood that embodiments disclosed herein can also include non-rectangular cathodic and anodic pulses.

FIG. 2A illustrates an example of a cathodic monophasic electrical current pulse. FIG. 2B illustrates an example of an anodic monophasic electrical current pulse. Because each of the monophasic electrical current pulses shown in FIGS. 2A-2B is not charged-balanced, each of the monophasic electrical current pulses of FIGS. 2A-2B can damage the tissue of a recipient.

Each of FIGS. 2C-2H depicts an example of a signal that is charge-balanced. In a charge-balanced signal, the amount of negative charge in the signal is equal to the amount of positive charge in the signal. FIG. 2C illustrates an example of a cathodic pseudo-monophasic signal that has a first phase, high amplitude, and short duration cathodic pulse followed by a second phase, low amplitude, and long duration anodic pulse. FIG. 2D illustrates an example of an anodic pseudo-monophasic signal that has a first phase, high amplitude, and short duration anodic pulse followed by a second phase, low amplitude, and long duration cathodic pulse.

FIG. 2E illustrates an example of a cathodic triphasic signal that has a first phase, low amplitude, and short duration anodic pulse followed by a second phase, high amplitude, and short duration cathodic pulse followed by a third phase, low amplitude, and short duration anodic pulse. FIG. 2F illustrates an example of an anodic triphasic signal that has a first phase, low amplitude, and short duration cathodic pulse followed by a second phase, high amplitude, and short duration anodic pulse followed by a third phase, low amplitude, and short duration cathodic pulse.

Each of the cathodic and anodic pseudo-monophasic signals in FIGS. 2C-2D and each of the cathodic and anodic triphasic signals in FIGS. 2E-2F has an asymmetric (i.e., non-symmetric) waveform, in that the cathodic pulse or pulses in each of these signals do not have the same inverted shape as the anodic pulse or pulses in the same signal. For example, the cathodic pseudo-monophasic signal in FIG. 2C is asymmetric, because the first phase cathodic pulse has a high amplitude and short duration and the second phase anodic pulse has a low amplitude and long duration.

FIG. 2G illustrates an example of a cathodic first biphasic signal that has a first phase, high amplitude, and short duration cathodic pulse followed by a second phase, high amplitude, and short duration anodic pulse. FIG. 2H illustrates an example of an anodic first biphasic signal that has a first phase, high amplitude, and short duration anodic pulse followed by a second phase, high amplitude, and short duration cathodic pulse. The signals shown in FIGS. 2G-2H are examples of biphasic electrical pulses that can be used in cochlear implant systems to generate the perception of hearing in a recipient.

Each of the cathodic and anodic pseudo-monophasic signals of FIGS. 2C-2D, each of the cathodic and anodic triphasic signals of FIGS. 2E-2F, and each of the cathodic and anodic biphasic signals of FIGS. 2G-2H is charge-balanced. Also, each of the 6 signals shown in FIGS. 2C, 2D, 2E, 2F, 2G, and 2H is a multiphasic signal, because each of these 6 signals has at least one cathodic pulse during one phase and an anodic pulse during another phase.

The present inventors have realized that the principle of polarity sensitivity can practically be used to estimate neural health based on the difference between cathodic pulses and anodic pulses that are applied to the auditory nerve of a recipient. The concept of polarity sensitivity is based on the observation that auditory nerve fibers (ANFs) are usually stimulated more effectively by cathodic pulses than anodic pulses. Based on the polarity sensitivity of ANFs, the auditory nerve of a recipient can be stimulated with different polarities (e.g., using a cochlear implant system). The response of the auditory nerve can then be recorded, and the survival of ANFs of the recipient can be estimated. The polarity sensitivity of ANFs is a new approach to evaluating neural health of a recipient.

According to some embodiments disclosed herein, systems and methods are provided for delivering cathodic and/or anodic asymmetric multiphasic stimulation to a recipient experiencing tinnitus, assessing responses from the recipient to the cathodic and/or anodic asymmetric multiphasic stimulation, and providing the asymmetric multiphasic stimulation determined to be more effective to an ear of the recipient to affect (e.g., treat) the tinnitus. The asymmetric multiphasic stimulation can include, for example, cathodic pseudo-monophasic signals, anodic pseudo-monophasic signals, cathodic triphasic signals, or anodic triphasic signals.

In some embodiments disclosed herein, a diagnostic tool can be used to characterize the neural heath and sensitivity of a recipient to cathodic and anodic asymmetric multiphasic stimulation. In these embodiments, cathodic and anodic asymmetric multiphasic stimulation is delivered to a recipient. Following the delivery of each type of stimulation (i.e., anodic or cathodic asymmetric multiphasic stimulation), responses from the recipient to the stimulation are collected. The responses of the recipient to the cathodic asymmetric multiphasic stimulation can be compared to the responses of the recipient to the anodic asymmetric multiphasic stimulation. Comparing the unique responses of a recipient to each of the cathodic and anodic asymmetric multiphasic stimulation can provide insights to the extent to which central or peripheral neural factors may be contributing to tinnitus or other dysfunctions in the recipient, such as for example, Parkinson's disease, Meniere's disease, dizziness, hyperacusis, migraine, etc.

The responses of the recipient to each type of stimulation can be used to develop a prognosis and/or treatment plan for any existing or future tinnitus disease, other auditory dysfunction, or other type of dysfunction in the recipient. The prognosis and/or treatment plan for the recipient can be used for programming and/or controlling a stimulation device that is adapted for affecting (e.g., treating) tinnitus, an auditory dysfunction, or other dysfunction in the recipient. The stimulation device can deliver the stimulation (e.g., anodic or cathodic asymmetric multiphasic stimulation) that is determined to be more effective to the recipient for treatment of tinnitus or other dysfunction.

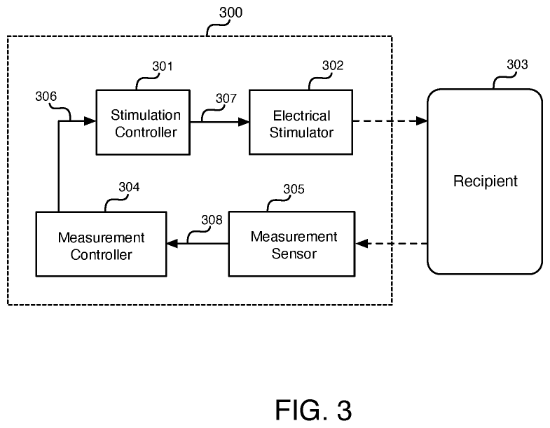

FIG. 3 is a diagram that depicts an example of a system 300 for evaluating and treating dysfunction in a recipient, according to an embodiment. The system 300 of FIG. 3 includes a stimulation controller 301, an electrical stimulator 302, a measurement controller 304, and a measurement sensor 305. Reference numeral 303 in FIG. 3 depicts a recipient or a portion of a body of a recipient (e.g., a cochlea of a recipient). The system 300 of FIG. 3 can be used in any type of medical device. For example, the system 300 can be used in hearing prostheses, including acoustic hearing aids, cochlear implants, bone conduction devices, middle ear auditory prostheses, direct acoustic stimulators, other electrically stimulating auditory prostheses (e.g., auditory brain stimulators), etc. The system 300 of FIG. 3 can also be used in or with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, brain stimulators, etc.

The electrical stimulator 302 is a device that can generate one or more electrical signals (i.e., stimulation) for delivery to recipient 303. For example, the electrical stimulator 302 can deliver any charge-balanced stimulation (e.g., cathodic and/or anodic asymmetric multiphasic stimulation or biphasic stimulation) to recipient 303 for evaluation, diagnosis, and/or treatment of a dysfunction, such as tinnitus. Stimulation controller 301 controls electrical stimulator 302 by providing one or more control signals 307 to electrical stimulator 302 that cause electrical stimulator 302 to deliver charge-balanced stimulation (e.g., cathodic and/or anodic asymmetric multiphasic stimulation or biphasic stimulation) to recipient 303. As examples that are not intended to be limiting, the electrical stimulator 302 can include an electrode in a cochlear implant system that is implanted in a cochlea of a recipient (e.g., as shown in FIGS. 1A-1B), or an electrode that is external to the cochlea of a recipient.

As an example of an embodiment in which the electrical stimulator 302 includes one or more electrodes in a cochlear implant system, the electrical stimulator 302 can include one or more of the stimulating contacts 126 of the cochlear implant system 100 of FIGS. 1A-1B. In this example, the processing module 125 is configured to generate a stimulation control signal 136 indicative of stimulation (e.g., cathodic and/or anodic asymmetric multiphasic stimulation) in response to input from electrophysiological response measurement system 160. In this example, the stimulation controller 301 can include processing module 125 and/or electrophysiological response measurement system 160. The stimulation control signal 136 is provided from processing module 125 to the RF transceiver 121, which transfers the stimulation control signal 136 (e.g., in an encoded manner) to the implantable component 104 via external coil 106 and implantable coil 122. The RF interface circuitry 124 receives the stimulation control signal 136 via implantable coil 122 and provides the stimulation control signal 136 to the stimulator unit 120. The stimulator unit 120 is configured to utilize the stimulation control signal 136 to generate electrical stimulation signals that are indicative of the stimulation. These electrical stimulation signals are transmitted to the one or more stimulating contacts 126 (e.g., electrodes). The stimulating contacts 126 provide the stimulation (e.g., the cathodic and/or anodic asymmetric multiphasic stimulation) to the cochlea of the recipient based on the electrical stimulation signals received from stimulator unit 120.

The measurement sensor 305 measures one or more electric potentials from the recipient 303 that are evoked in response to the stimulation (e.g., the cathodic and/or anodic asymmetric multiphasic stimulation) generated by electrical stimulator 302. In some embodiments that are provided as examples and are not intended to be limiting, the measurement sensor 305 can measure the electric potentials by performing an electrocochleography (ECochG) measurement from the recipient 303 after the stimulation (e.g., cathodic and/or anodic asymmetric multiphasic stimulation) is provided to the recipient 303.

Electrocochleography (ECochG) testing is a clinical technique that can be used, for example, to assess the residual hearing of a recipient suffering from partial hearing loss. In these embodiments, the measurement sensor 305 can include one or more electrodes that measure the electric potentials from the ear of the recipient 303 and that are external to a cochlea of the recipient. The electrodes can, for example, be used to implement the ECochG measurement. In these embodiments, the electrodes in the measurement sensor 305 can be, for example, invasive electrodes, such as electrodes in transtympanic (TT) needles, or non-invasive electrodes, such as extratympanic (ET) electrodes.

In other embodiments that are provided as examples and are not intended to be limiting, the measurement sensor 305 can measure electric potentials by performing an electroencephalogram (EEG) measurement that detects abnormalities in brain waves of the recipient 303 after stimulation (e.g., cathodic and/or anodic asymmetric multiphasic stimulation) is provided to the recipient 303. In these embodiments, the electrical stimulator 302 can deliver the stimulation (e.g., cathodic and/or anodic asymmetric multiphasic stimulation) to recipient 303 for evaluation, diagnosis, and/or treatment of a brain dysfunction in the recipient.

In still other embodiments that are provided as examples and are not intended to be limiting, the measurement sensor 305 can include one or more electrodes in a cochlear implant system that is implanted in a cochlea of recipient 303. In these embodiments, the measurement sensor 305 can include one or more of the stimulating contacts 126 (e.g., electrodes) of the cochlear implant system 100 of FIGS. 1A-1B, or the stimulating contacts of any other type of ear implant. In the example of FIGS. 1A-1B, the stimulating contacts 126 can measure electric potentials in the cochlea of the recipient (e.g., from hair cells) that are evoked in response to stimulation (e.g., cathodic and/or anodic asymmetric multiphasic stimulation) generated by the electrical stimulator 302. The electric potentials measured by the stimulating contacts 126 are delivered as one or more signals through lead region 116 to the RF interface circuitry 124 or other receiving circuitry in housing 115. The signals indicative of the measured electric potentials are then transmitted from the receiving circuitry in housing 115 to the implantable coil 122. The implantable coil 122 then transmits the signals indicative of the measured electric potentials through a wire and/or wirelessly to the RF transceiver 121 via external coil 106. The RF transceiver 121 then transmits the signals indicative of the electric potentials measured from the recipient to the processing module 125 for processing and analysis or externally. For example, the signals indicative of the electric potentials measured from the recipient can be transmitted to electrophysiological response measurement system 160 for processing and analysis.

The measurement sensor 305 is coupled to the measurement controller 304. The measurement controller 304 is configured to receive signals 308 from measurement sensor 305 that are indicative of the electric potentials measured from the recipient 303 in response to stimulation (e.g., cathodic and/or anodic asymmetric multiphasic stimulation) that was provided by stimulator 302 to recipient 303 to diagnosis a dysfunction (such as tinnitus). The measurement controller 304 can be used, for example, to evaluate the effectiveness of the cathodic and/or anodic asymmetric multiphasic stimulation for treatment of the dysfunction based on the electric potentials indicated by signals 308. As an example, measurement controller 304 can be used to determine, display, and/or evaluate the auditory neuro-phonic and/or cochlear microphonic of the recipient based on the electric potentials indicated by signals 308. The measurement controller 304 can, for example, include processing module 125 and/or a separate device, such as electrophysiological response measurement system 160. The measurement controller 304 can, for example, be part of a system that is configured to perform an electrocochleography (ECochG) measurement.

The electric potentials measured from the recipient 303 are processed by the measurement controller 304 (or another system) and are used to generate input 306. The input 306 can be provided to stimulation controller 301 manually or automatically by signals provided through electrical connections. The stimulation controller 301 uses the input 306 to generate the control signals 307 indicative of the stimulation (e.g., the cathodic and/or anodic asymmetric multiphasic stimulation). The control signals 307 are provided to the electrical stimulator 302.

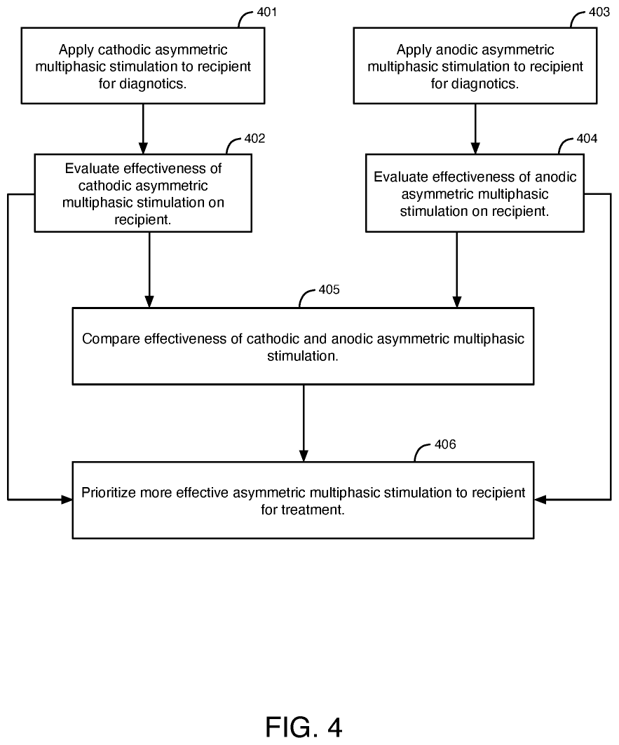

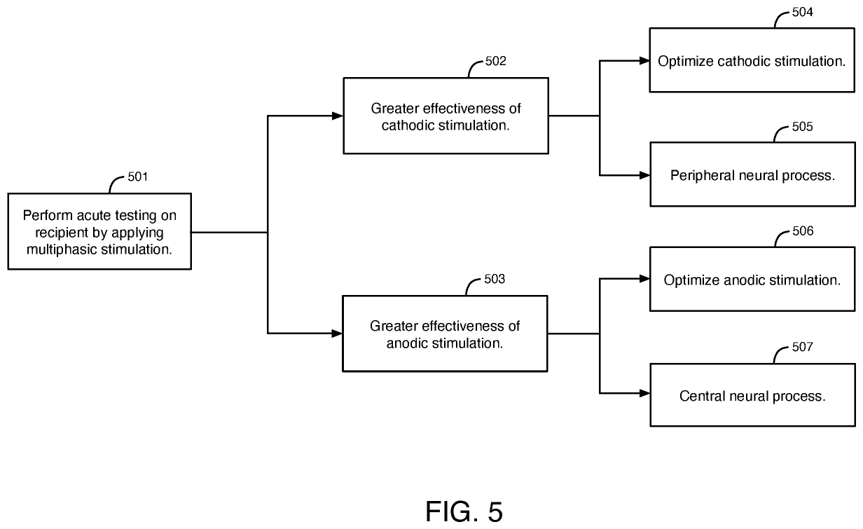

Electrical stimulator 302 provides the stimulation (e.g., the cathodic and/or anodic asymmetric multiphasic stimulation), indicated by the control signals 307 generated by the measurement controller 301, to the recipient 303 to affect (e.g., treat) a dysfunction in the recipient, such as tinnitus. For example, electrical stimulator 302 can deliver cathodic and/or anodic asymmetric multiphasic stimulation to the recipient 303 to mask tinnitus in the recipient, or in some cases, to reverse the pathophysiology of tinnitus. Further details of exemplary operations of the components of system 300 are described below with respect to FIGS. 4-5.

FIG. 4 depicts a flow chart that illustrates examples of operations that can be performed to test, diagnose, and provide treatment to a recipient for a dysfunction using asymmetric multiphasic stimulation. The operations of FIG. 4 can be used to monitor the neural health of a recipient by initially providing asymmetric multiphasic stimulation to the recipient to evaluate a dysfunction in the recipient, such as tinnitus. The responses of the recipient to the asymmetric multiphasic stimulation are evaluated for effectiveness for potential treatment of the dysfunction. The more effective asymmetric multiphasic stimulation is then applied to the recipient to treat the dysfunction. The treatment provided to the recipient can be customized based on the evaluation made using responses of the recipient to the asymmetric multiphasic stimulation. The operations of FIG. 4 are described herein primarily in the context of system 300 of FIG. 3. However, it should be understood that the operations of FIG. 4 can be performed using other types of systems.

In operation 401, cathodic asymmetric multiphasic (electrical) stimulation is applied to the recipient to perform diagnostics. For example, the electrical stimulator 302 of system 300 can apply the cathodic asymmetric multiphasic stimulation to recipient 303 in operation 401, as disclosed herein, for example, with respect to FIG. 3. The cathodic asymmetric multiphasic stimulation can, for example, be applied in operation 401 either intracochlear using a cochlear implant system or extracochlear.

In operation 403, anodic asymmetric multiphasic (electrical) stimulation is applied to the recipient to perform diagnostics. For example, the electrical stimulator 302 can apply the anodic asymmetric multiphasic stimulation to recipient 303 in operation 403, as disclosed herein, for example, with respect to FIG. 3. The anodic asymmetric multiphasic stimulation can, for example, be applied in operation 403 either intracochlear using a cochlear implant system or extracochlear.

The cathodic asymmetric multiphasic stimulation and the anodic asymmetric multiphasic stimulation can be applied to the recipient separately or concurrently in operations 401 and 403. If applied separately, the cathodic and the anodic asymmetric multiphasic stimulation can be applied to the recipient in any order (e.g., the cathodic asymmetric multiphasic stimulation can be applied first before the anodic asymmetric multiphasic stimulation). The cathodic and the anodic asymmetric multiphasic stimulation can be applied to the recipient, for example, using focused multipolar stimulation. The cathodic and the anodic asymmetric multiphasic stimulation can be applied to the recipient in operations 401 and 403 for any suitable time period in any suitable setting. For example, each of the cathodic asymmetric multiphasic stimulation and the anodic asymmetric multiphasic stimulation can be applied to the recipient for several minutes during a clinical consultation.

In operation 402, the effectiveness of the cathodic asymmetric multiphasic stimulation on the recipient is evaluated for the potential treatment of a dysfunction, such as tinnitus. For example, one or more responses of the recipient to the cathodic asymmetric multiphasic stimulation applied in operation 401 can be measured by the measurement sensor 305, provided to the measurement controller 304 in signals 308, and evaluated using measurement controller 304 in operation 402 for the potential treatment of a dysfunction in the recipient.

In operation 404, the effectiveness of the anodic asymmetric multiphasic stimulation on the recipient is evaluated for the potential treatment of a dysfunction, such as tinnitus. For example, one or more responses of the recipient to the anodic asymmetric multiphasic stimulation applied in operation 403 can be measured by the measurement sensor 305, provided to the measurement controller 304 in signals 308, and evaluated using measurement controller 304 in operation 404 for the potential treatment of a dysfunction in the recipient.

In operation 405, the effectiveness of the cathodic asymmetric multiphasic stimulation is compared with the effectiveness of the anodic asymmetric multiphasic stimulation. Electrical stimulator 302 and stimulation controller 301 can, for example, keep the current level and pulse width of the cathodic asymmetric multiphasic stimulation applied in operation 401 the same as the current level and pulse width of the anodic asymmetric multiphasic stimulation applied in operation 403 so that the effectiveness of each type of stimulation can be compared.

In order to compare the effectiveness of the cathodic and anodic asymmetric multiphasic stimulation in operation 405, a clinician can, for example, ask the recipient for the recipient's subjective response to each of the cathodic and anodic asymmetric multiphasic stimulation applied in operations 401 and 403. If the recipient generally reports perceiving greater effectiveness with the anodic asymmetric multiphasic stimulation, then the electrical stimulator 302 can be programmed by stimulation controller 301 to prioritize providing anodic asymmetric multiphasic stimulation to the recipient over cathodic asymmetric multiphasic stimulation (e.g., using the signal shown in FIG. 2D or 2F) in operation 406. If the recipient generally reports perceiving greater effectiveness with the cathodic asymmetric multiphasic stimulation, then the electrical stimulator 302 can be programmed by stimulation controller 301 to prioritize providing cathodic asymmetric multiphasic stimulation to the recipient over anodic asymmetric multiphasic stimulation (e.g., using the signal shown in FIG. 2C or 2E) in operation 406.

Alternatively, or in addition to receiving subjective responses from the recipient, measurement controller 304 in system 300 can objectively compare the effectiveness of the anodic asymmetric multiphasic stimulation to the effectiveness of the cathodic asymmetric multiphasic stimulation at alleviating the dysfunction (e.g., the tinnitus) in the recipient in operation 405. If measurement controller 304 determines that the anodic asymmetric multiphasic stimulation provides greater effectiveness at treating the dysfunction, then the electrical stimulator 302 can be programmed by stimulation controller 301 to prioritize providing anodic asymmetric multiphasic stimulation (e.g., using the signal shown in FIG. 2D or 2F) to the recipient over cathodic asymmetric multiphasic stimulation in operation 406. If measurement controller 304 determines that the cathodic asymmetric multiphasic stimulation provides greater effectiveness at treating the dysfunction, then the electrical stimulator 302 can be programmed by stimulation controller 301 to prioritize providing cathodic asymmetric multiphasic stimulation (e.g., using the signal shown in FIG. 2C or 2E) to the recipient over anodic asymmetric multiphasic stimulation in operation 406.

The prioritization of anodic asymmetric multiphasic stimulation in operation 406 can be achieved, in one example, by delivering a greater amplitude of anodic stimulation for a shorter time duration, followed by a lower amplitude of cathodic stimulation over a longer time duration, for example, as in the anodic pseudo-monophasic signal in FIG. 2D. The prioritization of cathodic asymmetric multiphasic stimulation in operation 406 can be achieved, in one example, by delivering a greater amplitude of cathodic stimulation for a shorter time duration, followed by a lower amplitude of anodic stimulation over a longer time duration, for example, as in the cathodic pseudo-monophasic signal in FIG. 2C. As discussed above, both cathodic and anodic pulses are used in one signal to achieve charge balancing in order to avoid tissue damage in a recipient. In other examples, the prioritization of the cathodic or anodic asymmetric multiphasic stimulation in operation 406 can be achieved using cathodic or anodic triphasic signals, as shown in FIGS. 2E and 2F, respectively.

In embodiments in which a recipient has a cochlear implant system, the prioritized stimulation (cathodic or anodic) provided for treatment of tinnitus using the cochlear implant system in operation 406 may have a different pole than the preferred stimulation (cathodic or anodic) for generating hearing perception using the cochlear implant system. In these embodiments, the cochlear implant system can apply stimulation for tinnitus to the cochlea of the recipient whenever the cochlear implant system is not needed for hearing, or when there is otherwise a greater need for tinnitus treatment than hearing treatment.

FIG. 5 depicts a graphical diagram that shows a process for providing multiphasic stimulation to a recipient for assessing and treating a dysfunction in the recipient. In operation 501, acute testing is performed on a recipient by applying multiphasic stimulation to the recipient. As examples, the multiphasic stimulation applied in operation 501 can be cathodic and anodic asymmetric multiphasic stimulation, such as cathodic and anodic pseudo-monophasic stimulation or cathodic and anodic triphasic stimulation. As another example, the multiphasic stimulation applied in operation 501 can be cathodic and anodic biphasic stimulation. The multiphasic stimulation can, for example, be applied in operation 501 using electrical stimulator 302 in system 300 or using another type of system.

A determination is then made (e.g., using system 300) as to whether the anodic stimulation or the cathodic stimulation provides greater effectiveness at treating the dysfunction (e.g., tinnitus). If the cathodic stimulation is determined to provide greater effectiveness at treating the dysfunction in operation 502, then the cathodic stimulation is optimized in operation 504. Optimizing the cathodic stimulation can, for example, include prioritizing cathodic stimulation to the recipient over anodic stimulation. Prioritizing the cathodic stimulation can include, for example, providing cathodic pulses that have a higher amplitude and a shorter duration than the anodic pulses in the signal (e.g., as in the signals shown in FIGS. 2C and 2E). Cathodic stimulation that provides greater effectiveness at treating neural dysfunction in a recipient (such as tinnitus) is often indicative of the neural dysfunction having a peripheral neural process, as indicated by box 505 in FIG. 5.

If the anodic stimulation is determined to provide greater effectiveness at treating the dysfunction in operation 503, then the anodic stimulation is optimized in operation 506. Optimizing the anodic stimulation can, for example, include prioritizing anodic stimulation to the recipient over cathodic stimulation. Prioritizing the anodic stimulation can include, for example, providing anodic pulses that have a higher amplitude and a shorter duration than the cathodic pulses in the signal (e.g., as in the signals shown in FIGS. 2D and 2F). Anodic stimulation that provides greater effectiveness at treating neural dysfunction in a recipient (such as tinnitus) is often indicative of the neural dysfunction having a central neural process, as indicated by box 507 in FIG. 5. The neural dysfunction treated with the operations of FIG. 5 can be any type of neural dysfunction.

If neither cathodic stimulation nor anodic stimulation provides a benefit in treating the dysfunction (e.g., tinnitus or other neural dysfunction) in the recipient, then multiphasic stimulation may be dismissed as a treatment for the dysfunction, and further tests can be performed to determine the origin of the dysfunction.

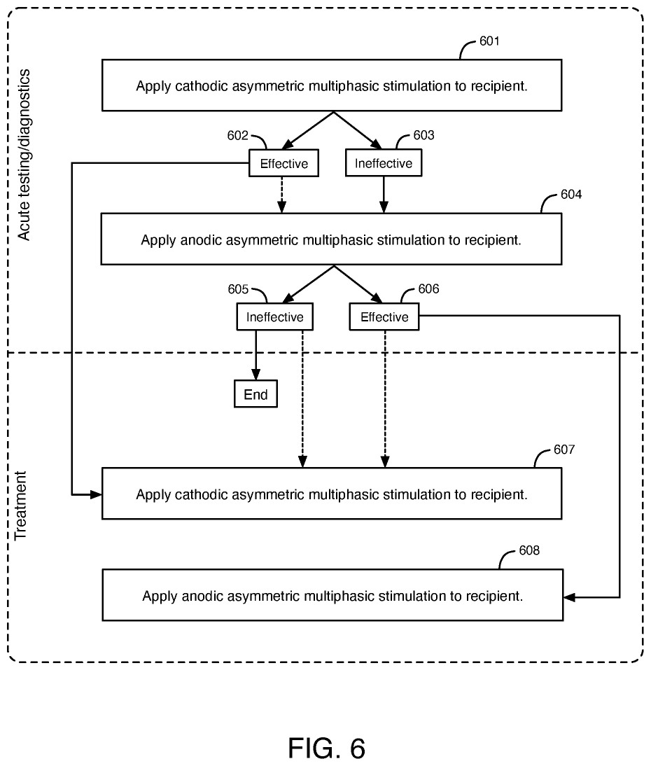

FIG. 6 depicts a flow chart that illustrates other examples of operations that can be performed to test, diagnose, and treat a recipient for a dysfunction using asymmetric multipolar multiphasic stimulation. The dysfunction can be, for example, tinnitus or another type of neural dysfunction. The operations of FIG. 6 are described herein primarily in the context of system 300 of FIG. 3. However, it should be understood that the operations of FIG. 6 can be performed using other types of systems.

Operations 601-606 of FIG. 6 are performed during acute testing and diagnosis of the dysfunction in the recipient, and operations 607-608 of FIG. 6 are performed during treatment of the dysfunction. Operations 601, 604, and 607-608 can be performed using intracochlear electrical stimulation (e.g., with a cochlear implant system) or using extracochlear electrical stimulation (e.g., with extracochlear electrodes). The operations of FIG. 6 can be applied to test, diagnose, and treat a recipient with tinnitus or another type of dysfunction.

In operation 601, cathodic asymmetric multiphasic stimulation is applied to the recipient to test and diagnose the dysfunction. As an example, the electrical stimulator 302 of system 300 can apply the cathodic asymmetric multiphasic stimulation to recipient 303 in operation 601, as disclosed herein, for example, with respect to FIG. 3. The cathodic asymmetric multiphasic stimulation applied in operation 601 can be, for example, pseudo-monophasic or triphasic stimulation.

Subsequently, a determination is made as to whether the cathodic asymmetric multiphasic stimulation applied to the recipient in operation 601 was effective (602) or ineffective (603) at treating the dysfunction using subjective or objective measurements, such as the measurements described above with respect to FIGS. 3-5. If the cathodic asymmetric multiphasic stimulation applied in operation 601 was effective (602) at treating the dysfunction during acute testing and diagnosis, then cathodic asymmetric multiphasic stimulation is applied to the recipient as an on-going treatment for the dysfunction in operation 607. The cathodic asymmetric multiphasic stimulation applied in operation 607 can be, for example, pseudo-monophasic or triphasic stimulation.

If the cathodic asymmetric multiphasic stimulation applied in operation 601 was ineffective (603) at treating the dysfunction, then anodic asymmetric multiphasic stimulation is applied to the recipient to test and diagnose the dysfunction in operation 604. Anodic asymmetric multiphasic stimulation can also be applied to the recipient to test and diagnose the dysfunction in operation 604, even if the cathodic asymmetric multiphasic stimulation applied in operation 601 was effective, as shown by the dotted arrow from 602 in FIG. 6, for comparison purposes. As an example, the electrical stimulator 302 of system 300 can apply the anodic asymmetric multiphasic stimulation to recipient 303 in operation 604, as disclosed herein, for example, with respect to FIG. 3. The anodic asymmetric multiphasic stimulation applied in operation 604 can be, for example, pseudo-monophasic or triphasic stimulation.

Subsequently, a determination is made as to whether the anodic asymmetric multiphasic stimulation applied to the recipient in operation 604 was effective (606) or ineffective (605) at treating the dysfunction using subjective or objective measurements, such as the measurements described above with respect to FIGS. 3-5. If the anodic asymmetric multiphasic stimulation was effective (606) at treating the dysfunction in operation 604 during acute testing and diagnosis, then anodic asymmetric multiphasic stimulation is applied to the recipient as an on-going treatment for the dysfunction in operation 608. The anodic asymmetric multiphasic stimulation applied in operation 608 can be, for example, pseudo-monophasic or triphasic stimulation.

If the anodic asymmetric multiphasic stimulation applied in operation 604 was ineffective (605) at treating the dysfunction, and the cathodic asymmetric multiphasic stimulation applied in operation 601 was also ineffective (603) at treating the dysfunction, then the process of FIG. 6 ends without treatment. If the anodic asymmetric multiphasic stimulation applied in operation 604 was less effective at treating the dysfunction than the cathodic asymmetric multiphasic stimulation applied in operation 601, then cathodic asymmetric multiphasic stimulation is applied to the recipient as an on-going treatment for the dysfunction in operation 607, as shown by the dotted arrows from 605-606 to 607 in FIG. 6.

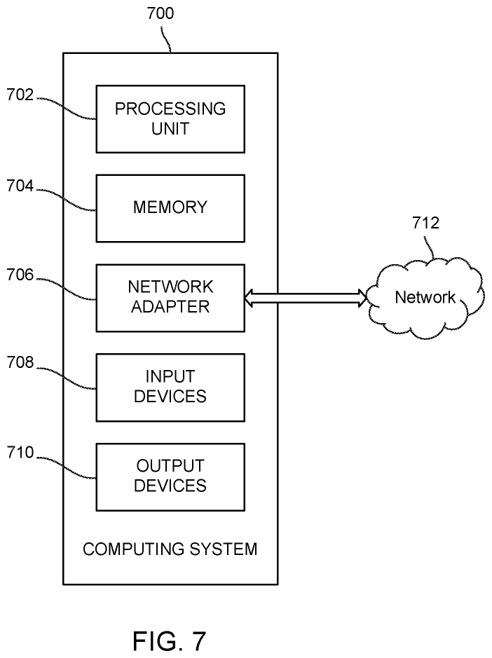

FIG. 7 illustrates an example of a computing system 700 within which one or more of the disclosed embodiments can be implemented. For example, computing system 700 can include the stimulation controller 301 and the measurement controller 304 of FIG. 3 and/or system 160. The computing system 700 can, for example, be used to determine the type of stimulation to apply to a recipient to treat a dysfunction, such as tinnitus, as disclosed herein with respect to FIGS. 2A-6.

Computing systems, environments, or configurations that can be suitable for use with examples described herein include, but are not limited to, personal computers, server computers, hand-held devices, laptop devices, multiprocessor systems, microprocessor-based systems, programmable consumer electronics (e.g., smart phones), network computers, minicomputers, mainframe computers, tablets, distributed computing environments that include any of the above systems or devices, and the like. The computing system 700 can be a single virtual or physical device operating in a networked environment over communication links to one or more remote devices. The remote device can be an auditory prosthesis (e.g., the cochlear implant system of FIGS. 1A-1B), a personal computer, a server, a router, a network personal computer, a peer device or other common network node.

Computing system 700 includes at least one processing unit 702 and memory 704. The processing unit 702 includes one or more hardware or software processors (e.g., Central Processing Units) that can obtain and execute instructions. The processing unit 702 can communicate with and control the performance of other components of the computing system 700. The memory 704 is one or more software-based or hardware-based computer-readable storage media operable to store information accessible by the processing unit 702.

The memory 704 can store instructions executable by the processing unit 702 to implement applications or cause performance of operations described herein, as well as store other data. The memory 704 can be volatile memory (e.g., random access memory or RAM), non-volatile memory (e.g., read-only memory or ROM), or combinations thereof. The memory 704 can include transitory memory or non-transitory memory. The memory 704 can also include one or more removable or non-removable storage devices. In examples, the memory 704 can include non-transitory computer readable storage media, such as RAM, ROM, EEPROM (Electronically-Erasable Programmable Read-Only Memory), flash memory, optical disc storage, magnetic storage, solid state storage, or any other memory media usable to store information for later access. In examples, the memory 704 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 704 can include wired media such as a wired network or direct-wired connection, and wireless media such as acoustic, radio-frequency, infrared and other wireless media or combinations thereof.

In the illustrated example, the system 700 further includes a network adapter 706, one or more input devices 708, and one or more output devices 710. The system 700 can include other components, such as a system bus, component interfaces, a graphics system, a power source (e.g., a battery), among other components.

The network adapter 706 is a component of the computing system 700 that provides network access to network 712. The network adapter 706 can provide wired or wireless network access and can support one or more of a variety of communication technologies and protocols, such as ETHERNET, cellular, BLUETOOTH, near-field communication, and RF (Radiofrequency), among others. The network adapter 706 can include one or more antennas and associated components configured for wireless communication according to one or more wireless communication technologies and protocols.

The one or more input devices 708 are devices over which the computing system 700 receives input from a user. The one or more input devices 708 can include physically-actuatable user-interface elements (e.g., buttons, switches, or dials), touch screens, keyboards, mice, pens, and voice input devices, among others input devices.

The one or more output devices 710 are devices by which the computing system 700 is able to provide output to a user. The output devices 710 can include, displays, speakers, and printers, among other output devices.

Any embodiment or any feature disclosed herein can be combined with any one or more other embodiments and/or other features disclosed herein, unless explicitly indicated otherwise. Any embodiment or any feature disclosed herein can be explicitly excluded from use with any one or more other embodiments and/or other features disclosed herein, unless explicitly indicated otherwise. It is noted that any method detailed herein also corresponds to a disclosure of a device and/or system configured to execute one or more or all of the method actions associated with the device and/or system as detailed herein. It is further noted that any disclosure of a device and/or system detailed herein corresponds to a method of making and/or using that device and/or system, including a method of using that device according to the functionality detailed herein.

The foregoing description of the exemplary embodiments of the present invention has been presented for the purpose of illustration. The foregoing description is not intended to be exhaustive or to limit the present invention to the examples disclosed herein. In some instances, features of the present invention can be employed without a corresponding use of other features as set forth. Many modifications, substitutions, and variations are possible in light of the above teachings, without departing from the scope of the present invention.

1. A system comprising:
a stimulator configured to provide at least one asymmetric multiphasic stimulation to a recipient for affecting tinnitus in the recipient.

2. The system of claim 1, wherein the stimulator is further configured to provide at least one of a pseudo-monophasic signal or a triphasic signal to the recipient as the at least one asymmetric multiphasic stimulation for affecting the tinnitus.

3. The system of claim 1, wherein the stimulator is further configured to provide at least one of an electrical cathodic asymmetric multiphasic stimulation or an electrical anodic asymmetric multiphasic stimulation to the recipient as the at least one asymmetric multiphasic stimulation for affecting the tinnitus.

4. The system of claim 1 further comprising:
a measurement sensor configured to measure at least one electric potential evoked from the recipient in response to the at least one asymmetric multiphasic stimulation provided by the stimulator.

5. The system of claim 1 further comprising:
a measurement controller configured to evaluate an effectiveness of the at least one asymmetric multiphasic stimulation for treatment of the tinnitus based on at least one electric potential measured from the recipient in response to the at least one asymmetric multiphasic stimulation provided by the stimulator.

6. The system of claim 5, wherein the measurement controller is further configured to compare an effectiveness of cathodic asymmetric multiphasic stimulation on the tinnitus to an effectiveness of anodic asymmetric multiphasic stimulation on the tinnitus.

7. The system of claim 1 further comprising:
a stimulation controller configured to control the stimulator by causing the stimulator to deliver the at least one asymmetric multiphasic stimulation to the recipient.

8. The system of claim 1, wherein the stimulator is further configured to provide a cathodic asymmetric multiphasic stimulation and an anodic asymmetric multiphasic stimulation to the recipient for evaluation of the tinnitus.

9. The system of claim 1, wherein the stimulator is further configured to treat the tinnitus by prioritizing one of cathodic asymmetric multiphasic stimulation or anodic asymmetric multiphasic stimulation that is determined to be more effective at treating the tinnitus.

10. A method comprising:
generating asymmetric multiphasic stimulation; and
providing the asymmetric multiphasic stimulation to an ear of a recipient to affect tinnitus in the recipient.

11. The method of claim 10, wherein providing the asymmetric multiphasic stimulation further comprises providing at least one of a cathodic asymmetric multiphasic stimulation or an anodic asymmetric multiphasic stimulation to the ear of the recipient using an electrical stimulator device.

12. The method of claim 11 further comprising:
receiving responses to the asymmetric multiphasic stimulation from the recipient; and
determining whether the cathodic asymmetric multiphasic stimulation or the anodic asymmetric multiphasic stimulation is more effective at treating the tinnitus in the recipient based on the responses.

13. The method of claim 12 further comprising:
treating the tinnitus by prioritizing the cathodic asymmetric multiphasic stimulation or the anodic asymmetric multiphasic stimulation that is determined to be more effective at treating the tinnitus.

14. The method of claim 10, wherein providing the asymmetric multiphasic stimulation further comprises providing pseudo-monophasic or triphasic stimulation to the ear of the recipient.

15. The method of claim 10 further comprising:
receiving responses to the asymmetric multiphasic stimulation from the recipient indicative of the tinnitus using electrocochleography.

16. The method of claim 10 further comprising:
providing an indication that the tinnitus comprises a peripheral neural process based on cathodic stimulation providing greater effectiveness at treating the tinnitus in the recipient than anodic stimulation.

17. The method of claim 10 further comprising:
providing an indication that the tinnitus comprises a central neural process based on anodic stimulation providing greater effectiveness at treating the tinnitus in the recipient than cathodic stimulation.

18. A non-transitory computer readable storage medium comprising computer readable instructions stored thereon for causing a computing system to:
provide cathodic stimulus comprising first non-symmetric pulses to a recipient;
provide anodic stimulus comprising second non-symmetric pulses to the recipient; and
compare a first effect of the cathodic stimulus with a second effect of the anodic stimulus on the recipient.

19. The non-transitory computer readable storage medium of claim 18, wherein each of the cathodic stimulus and the anodic stimulus comprises at least one of pseudo-monophasic or triphasic electrical stimulation.

20. The non-transitory computer readable storage medium of claim 18, wherein the computer readable instructions further cause the computing system to compare the first effect of the cathodic stimulus on tinnitus of the recipient with the second effect of the anodic stimulus on the tinnitus of the recipient.

21. (canceled)

22. (canceled)

23. (canceled)

24. (canceled)
